(19) United States
(12) Patent Application Publication
(10) Pub. No.: US 2005/0049293 A1
(43) Pub. Date: Mar. 3, 2005

(54) Use of cholinesterase antagonists to treat insulin resistance

(76) Inventor: W Wayne Lautt, Winnipeg (CA)

Correspondence Address: MERCHANT & GOULD PC, P.O. BOX 2903, MINNEAPOLIS, MN, 55402-0903, US

(21) Appl. No.: 10/502,066

(22) PCT Filed: Jan. 27, 2003
(86) PCT No.: PCT/CA03/00078

Related U.S. Application Data
(60) Provisional application No. 60/350,958, filed on Jan. 25, 2002.

(51) Int. Cl. A61K 31/407

(52) U.S. Cl. 514/411; 514/291

(57) ABSTRACT

There is provided a method of reducing insulin resistance in a mammalian subject comprising administering a suitable acetylcholine esterase antagonist.

This application claims priority of invention from U.S. Patent Application No. 60/350,958, filed 25 Jan. 2002.

### FIELD OF THE INVENTION

The invention relates to the field of treatments for insulin resistance.

### BACKGROUND

Insulin resistance is a significant health challenge for a wide range of patients, including those with type II diabetes, metabolic obesity, and various liver conditions.

The picture that is emerging is one of complex multiple interacting systems with reflex parasympathetic effects in the liver capable of causing more than one reaction and of triggering reactions in other organs.

In fasted cats, the hypoglycemic response to a bolus administration of insulin was reduced by 37% by hepatic denervation. These cats developed insulin resistance immediately following acute denervation of the liver. The degree of reduction of response to insulin was maximal after anterior plexus denervation and did not increase further with addition of denervation of the posterior nerve plexus or bilateral vagotomy thus demonstrating that all of the nerves of relevance were in the anterior plexus. To avoid the complexity of the reaction to hypoglycemia, the rapid insulin sensitivity test (RIST) was employed (Lautt et al., Can. J. Physiol. Pharmacol. 76:1080 (1998)) wherein a euglycemic clamp was used following the administration of insulin and the response was quantitated as the amount of glucose required to be infused over the test period in order to hold arterial blood glucose levels constant. The RIST methodology has been published in detail and has been demonstrated in both cats and rats. It is highly reproducible. Insulin, glucagon, and catecholamine levels remain unchanged between tests.

Cats showed a dose-related development of insulin resistance using atropine (a cholinergic muscarinic receptor antagonist) that was of a similar magnitude to that produced by surgical denervation. The dose of atropine required to produce a full insulin resistance is 3 mg/kg (4 μmol/kg) administered into the portal vein. A similar degree of insulin resistance was achieved with 10⁻⁷ mmol/kg of the M₁ muscarinic selective antagonist, pirenzepine, and with 10⁻⁶ μmol/kg of the M₂ selective antagonist, methoctramine. Although not conclusive, the data suggest that the response may be mediated by the M₁ muscarinic receptor subtype.

Although the liver appeared to be the organ that produced the insulin resistance, it was not clear that the liver was the resistant organ. In order to determine the site of insulin resistance, a further series was done in cats that measured arterial-venous glucose responses across the hindlimbs, extrahepatic splanchnic organs, and liver. The intestine was unresponsive to the bolus insulin administration both before and after atropine or anterior plexus denervation or the combination of both. The hepatic response was also not notably altered whereas the glucose uptake across the hindlimbs, primarily representing skeletal muscle uptake, was decreased following atropine or hepatic parasympathetic denervation. These results indicated that interference with hepatic parasympathetic nerves led to insulin resistance in skeletal muscle.

It was further demonstrated that the same degree of resistance could be produced by pharmacological blockade of parasympathetic nerve function using the muscarinic receptor antagonist, atropine. Following a meal, insulin is released from the pancreas. The presence of insulin in the blood elicits a hepatic parasympathetic reflex that results in the release of acetylcholine in the liver that results in the generation and release of nitric oxide which acts to control the sensitivity of skeletal muscle to insulin through the action of a hormone released from the liver, a hepatic insulin sensitizing substance (HISS) which selectively stimulates glucose uptake and storage as glycogen in tissues including skeletal muscle.

In the absence of HISS, the large muscle mass is highly resistant to insulin and the glucose storage in skeletal muscle is severely reduced. Interruption of any part of the parasympathetic-mediated release of HISS results in insulin resistance. This parasympathetic reflex regulation of HISS release is a fundamental mechanism by which the body regulates responsiveness to insulin and this mechanism is adjusted according to the prandial state, that is, according to how recently there has been a consumption of nutrients.

In a fasted condition, HISS release in response to insulin is minimal or absent so that if insulin is released in this situation, there is a minimal metabolic effect. Following a meal, the parasympathetic reflex mechanism is amplified so that HISS release occurs and results in the majority of the ingested glucose stored in skeletal muscle.

The consequence of lack of HISS release is the absence of HISS which results in severe insulin resistance, referred to as HISS-dependent insulin resistance (“HDIR”). In this situation, the pancreas is required to secrete substantially larger amounts of insulin in order that the glucose in the blood is disposed of to prevent hyperglycemia from occurring. If this condition persists, insulin resistance will progress to a state of type 2 diabetes (non-insulin dependent diabetes mellitus) and eventually will lead to a complete exhaustion of the pancreas thus requiring the patient to resort to injections of insulin. Thus, it appears that any condition in which the hepatic parasympathetic reflex is dysfunctional will result in insulin resistance.

It is believed that the insulin resistance that is seen in a variety of conditions (non-insulin dependent diabetes, essential hypertension, obesity, chronic liver disease, fetal alcohol effects, old age, and chronic inflammatory diseases) represents a state of HDIR parasympathetic dysfunction. Lack of HISS would also be anticipated to result in obesity at the early stage of the resultant metabolic disturbance (the obese often become diabetic).

Normally after a meal, the liver takes up a small proportion of glucose and releases HISS to stimulate skeletal muscle to take up the majority of the glucose load. In the absence of HISS, the skeletal muscle is unable to take up the majority of glucose thus leaving the liver to compensate. The hepatic glycogen storage capacity is insufficient to handle all of the glucose, with the excess being converted to lipids which are then incorporated into lipoproteins and transported to adipose tissue for storage as fat. Provision of HISS to these individuals would restore the nutrition partitioning so that the nutrients are stored primarily as glycogen in the skeletal muscle rather than as fat in the adipose tissue.

Thus, it is an object of the invention to provide a method of reducing insulin resistance.

### SUMMARY OF THE INVENTION

Insulin resistance in skeletal muscle relating to insufficient response to the hepatic parasympathetic reflex can be alleviated by increasing the effect of released acetylcholine on hepatic muscarinic receptors. This can be accomplished by reducing the rate at which acetylcholine is broken down by acetylcholine esterase. Thus, in an embodiment of the invention there is provided the use of an acetylcholine esterase antagonist to reduce insulin resistance.

In an embodiment of the invention there is provided a method of reducing insulin resistance in a mammalian patient comprising administering a suitable cholinesterase antagonist.

In an embodiment of the invention there is provided a method of amplifying the effect of the hepatic parasympathetic reflex on skeletal muscle sensitivity comprising administering a cholinesterase antagonist.

### BRIEF DESCRIPTION OF THE FIGURES

FIG. 1 is a graphical representation of the effect of neostigmine, on the RIST index of rats given atropine.

FIG. 2 is a graphical depiction of the results of Example 2.

FIG. 3 is a graphical depiction of results of Example 3.

### DETAILED DESCRIPTION OF THE PREFERRED EMBODIMENTS

The weak response of isolated perfused livers to insulin appears secondary to the lack of hepatic parasympathetic innervation. This hypothesis is supported by the fact that glycogen synthase activity rapidly increases following vagus nerve or lateral hypothalamic stimulation. Acetylcholine or choline alone, or insulin alone, do not enhance the deposition of glycogen in isolated perfused rat liver, but stimulation of glycogen synthesis required the combined action of insulin plus cholinergic stimulation.

Direct electrical stimulation of the hepatic anterior nerve plexus in cats leads to a rapid decrease in glucose output reaching approximately 75% of maximal response by two minutes. No net increase in hepatic uptake by parasympathetic stimulation in fasted cats was observed.

In the isolated rat liver in non-fasted rats, electrical stimulation of parasympathetic nerves did not alter glucose or lactate metabolism unless insulin was simultaneously presented. While the parasympathetic nerves had a synergistic effect with insulin they were antagonistic to the glucose liberating effect of glucagon.

In both of the previous experiments, direct electrical stimulation of the parasympathetic nerves was demonstrable only after the sympathetic nerves had been eliminated. In the cat studies, the hepatic sympathetic nerves had been destroyed by prior administration of intraportal 6-hydroxydopamine whereas in the latter study the sympathetic nerves in the isolated rat liver were blocked using simultaneous administration of an alpha and beta adrenergic receptor blocker.

Thus, it appears that under conditions of elevated sympathetic nerve input or activation of glycogen phosphorylase above a certain threshold level, the hepatic parasympathetic nerves are without effect.

The amount of a glucose load taken up by the liver is highly dependent upon the route of glucose delivery to the liver. Intravenously administered glucose, even in the presence of hyperinsulinemia, resulted in the liver taking up less than 15% of the total glucose load. In dramatic contrast, after oral glucose administration at least 60% of the total glucose load was taken up by the liver. Orally consumed glucose may cause a hepatic parasympathetic reflex effect that enhances insulin-mediated glucose uptake by the liver.

Hepatic denervation eliminates the selective effect of portal glucose delivery on glucose uptake. This, and the demonstration that atropine similarly reduced the proportion of glucose sequestered by the liver following oral administration from 80% to 44%, suggested that hepatic parasympathetic nerves are involved with producing the selective hepatic uptake of glucose in response to oral or intraportal glucose loading.

The portal glucose signal appears to ordinarily be needed in order for the liver to respond effectively to insulin by producing glucose uptake. This effect can be blocked by administration of atropine to the liver and could be duplicated by the administration of acetylcholine thus identifying the process as acting through cholinergic receptors.

The above study was carried out in an isolated perfused liver preparation. Although the liver was perfused in situ, it is a reasonable assumption that no extrahepatic nerves retained function. One possible conclusion is that sensory nerves within the liver sense the glucose gradient and transmit the information by intrahepatic nerves releasing acetylcholine to act on muscarinic receptors. This would suggest a purely intrahepatic reflex system. This study is compatible with the study which found that hepatic denervation eliminated the selective effect of portal glucose delivery on glucose uptake if one assumes that intrahepatic nerves deteriorate with surgical denervation of the nerve trunk supplying the liver (since the surgical denervation was carried out three weeks prior to the experiment). This is the first data offering support for the existence of a reflex arc located entirely within the liver.

The efferent limb of this reflex appears to be dependent upon hepatic parasympathetic nerves. The afferent limb of the reflex appears to depend upon the presence of glucose receptors located in the portal vein. The nerve pathway does not pass through the CNS and may, in fact, be entirely intrahepatic.

The absorption of orally administered glucose in conscious dogs was suppressed and delayed by administration of atropine. The mechanism of this response has recently been demonstrated using an isolated, jointly perfused small bowel and liver preparation in rats. Administration of insulin into the portal blood supply leads to a parasympathetic nerve-mediated increase in absorption of glucose from the lumen of the intestine. The effect can be blocked by atropine and mimicked by carbachol. The afferent limb of the reflex is activated by insulin with receptors located in the portal vein or liver and the efferent limb represents muscarinic nerves supplying the intestine.

The neural pathway connecting the sensory and effector branches of the reflex is not known but, in this unique preparation, would likely occur through one of two sources. One route would be from the liver along the portal vein through the posterior hepatic plexus to the intestine. The other would involve transmission through the celiac ganglion which remained intact in this preparation. Regardless of the course, this is another example of a splanchnic reflex that does not pass through the central nervous system. This mechanism likely serves the function of assuring that maximum glucose absorption only occurs at a time when the organs sensitive to insulin-induced uptake have also been stimulated.

Cats showed a dose-related development of insulin resistance using atropine that was of a similar magnitude to that produced by surgical denervation. The dose of atropine required to produce a full insulin resistance is 3 mg/kg (4 μmol/kg) administered into the portal vein. A similar degree of insulin resistance was achieved with 10⁻⁷ mmol/kg of the M₁ muscarinic selective antagonist, pirenzepine, and with 10⁻⁶ μmol/kg of the M₂ selective antagonist, methoctramine. These data suggest that the response may be mediated by the M₁ muscarinic receptor subtype.

In order to determine the site of insulin resistance, a further series was done in cats that measured arterial-venous glucose responses across the hindlimbs, extrahepatic splanchnic organs, and liver. The intestine was unresponsive to the bolus insulin administration both before and after atropine or anterior plexus denervation or the combination of both. The hepatic response was also not notably altered whereas the glucose uptake across the hindlimbs, primarily representing skeletal muscle uptake, was decreased following atropine or hepatic parasympathetic denervation. These results indicated that interference with hepatic parasympathetic nerves can lead to insulin resistance in skeletal muscle.

It was further determined that the same degree of resistance could be produced by pharmacological blockade of parasympathetic nerve function using the muscarinic receptor antagonist, atropine. Following a meal, insulin is released from the pancreas. The presence of insulin in the blood elicits a hepatic parasympathetic reflex that results in the release of acetylcholine in the liver which results in the generation and release of nitric oxide which acts to control the sensitivity of skeletal muscle to insulin.

Acetylcholine infused directly into the portal vein (2.5 μg/kg/min) results in a complete reversal of the insulin resistance induced by surgical denervation. Administration of the same dose of acetylcholine intravenously produces no reversal. Intraportal administration directly targets the liver whereas intravenous infusion bypasses the liver and is not organ selective. This demonstration is extremely important in that the data suggest that the signal from the liver skeletal muscle is blood-borne.

While the invention is not limited to any particular mechanism of action, the model for insulin resistance which has emerged is that, in normal individuals, the eating of a meal results not only in the release of insulin, but also in a hepatic parasympathetic reflex. The hepatic parasympathetic effect results in the release of acetylcholine (ACh) which activates muscarinic receptors in the liver, leading to activation of hepatic nitric oxide synthase (NOS) and the generation of nitric oxide (NO), which in turn causes increased guanyl cyclase (GC) activity, resulting in increased levels of cyclic guanosine monophosphate (“cGMP”) and the release of a hepatic insulin sensitizing substance (HISS) into the blood which ultimately leads to an increase in insulin sensitivity in skeletal muscle.

In some instances, such as disease or injury, the release of acetylcholine by the hepatic parasympathetic neurons is impaired, and it may be desirable to enhance the effectiveness of the reduced amount which is present.

A method for enhancing the effectiveness of acetylcholine and the use of this method in the treatment of insulin resistance has been developed.

HISS-dependent insulin resistance (“HDIR”) is defined as a reduction in the response to insulin secondary to a failure of HISS action on glucose disposal. When insulin fails to result in HISS release from the liver or its action on skeletal muscle is otherwise impaired, a state of HDIR is said to exist. With a pure state of HDIR, the direct glucose uptake stimulation effect of insulin is not impaired.

During normal nervous system function, acetylcholine is broken down by acetylcholine esterase in the synaptic cleft. This prevents the unlimited build-up of acetylcholine in the synaptic cleft, which, in normal patients, could result in an undesirably high level of acetylcholine binding to its receptor long after the initial release of acetylcholine from the presynaptic terminal.

However, where acetylcholine production or release is below normal levels (or receptor levels on the post-synaptic neuron are unusually low), it may be desirable to increase the residency time of acetylcholine in the synaptic cleft, thereby allowing a greater interaction between acetylcholine and its receptors on the post-synaptic neuron and potentially amplifying its effects.

In one embodiment of the invention, an acetylcholine esterase antagonist is used to reduce the breakdown of acetylcholine in the hepatic parasympathetic nerve synapses. The precise dose of ACh esterase antagonist desirable will be determined by a number of factors which will be apparent to those skilled in the art, in light of the disclosure herein. In particular, the identity of the antagonist, the formulation and route of administration employed, the patient's gender, age and weight, as well as the extent of ACh production in the hepatic parasympathetic neurons, the number and effectiveness of the ACh receptors on the post-synaptic terminal and the severity of the condition to be treated will often be considered. Where it is impractical to conduct the tests necessary to determine the receptor abundance on the post-synaptic terminal and/or the other factors such as the extent of hepatic ACh production, the appropriate dose can be determined through the administration of a dose suitable for a majority of patients similar to the subject in respect of those factors which have been assessed with subsequent examination of insulin resistance and symptoms of excessive ACh esterase exposure.

A wide variety of acetylcholine esterase antagonists are known in the art and specifically contemplated for use in certain embodiments of the invention. By way of non-limiting example, donepezil, galantamine, rivastigme and tacrine are currently in therapeutic use for the treatment of Alzheimer's disease. If compounds such as those listed above were used to reduce insulin resistance they would preferably be targeted to the liver. Further non-limiting examples of acetylcholine esterase antagonists include physostigimine (eserine), edrophonium, demecarium, pyridostigmine, phospholine, metrifonate, neostigmine, galanthamine, zanapezil and ambenonium.

Any suitable acetylcholine esterase antagonist may be employed. An acetylcholine esterase antagonist will be “suitable” if: (a) at the dose and method of administration to the mammalian patient, it is not acutely toxic, and does not result in chronic toxicity disproportionate to the therapeutic benefit derived from treatment; and (b) at the dose and method of administration to the mammalian patient it reduces insulin resistance in the patient.

It is preferable to minimize the diffusion of the acetylcholine esterase into the spinal cord and brain.

In one embodiment, the acetylcholine esterase antagonist is preferentially targeted to the liver. Targeting of the antagonist to the liver can be accomplished through the use of any pharmaceutically acceptable liver-targeting substance. For example, it can be bound to albumin or bile salts for preferential delivery to liver. Alternatively, the antagonist may be incorporated into or encapsulated within liposomes which are preferentially targeted to the liver. In one embodiment, the antagonist is administered in a precursor form, and the precursor is selected to be metabolised to the active form by enzymes preferentially found in the liver.

In some instances it will be desired to prepare and administer a composition comprising an acetylcholine esterase antagonist and at least one other drug used in the treatment of diabetes. Examples of such drugs are listed in Table I.

TABLE I

| a. | Insulin and insulin analogues | |
|---|---|---|
| b. | Type II Diabetes drugs | |
| | i. | Sulfonylurea agents |

| | |
|---|---|
| 1. | First Generation |

| | |
|---|---|
| a. | Tolbutamide |
| b. | Acetohexamide |
| c. | Tolazamide |
| d. | Chlorpropamide |

| | |
|---|---|
| 2. | Second Generation |

| | |
|---|---|
| a. | Glyburide |
| b. | Glipizide |
| c. | Glimepiride |

| | |
|---|---|
| ii. | Biguanide agents |

| | |
|---|---|
| 1. | metformin |

| | |
|---|---|
| iii. | Alpha-glucosidase inhibitors |

| | |
|---|---|
| 1. | Acarbose |
| 2. | Miglitol |

| | |
|---|---|
| iv. | Thiazolidinedione Agents (insulin sensitizers) |

| | |
|---|---|
| 1. | Rosiglitazone |
| 2. | Pioglitazone |
| 3. | Troglitazone |

| | |
|---|---|
| v. | Meglitinide Agents |

| | |
|---|---|
| 1. | Repaglinide |

| c. | Phosphodiesterase Inhibitors | |
|---|---|---|
| | i. | Anagrelide |
| | ii. | Tadalafil |
| | iii. | Dipyridamole |
| | iv. | Dyphylline |
| | v. | Vardenafil |
| | vi. | Cilostazol |
| | vii. | Milrinone |
| | viii. | Theophylline |
| | ix. | Sildenafil |
| | x. | Caffeine |
| d. | Cholinergic Agonists | |
| | i. | Acetylcholine |
| | ii. | Methacholine |
| | iii. | Bethanechol |
| | iv. | Carbachol |
| | v. | Pilocarpine hydrochloride |
| e. | Nitric Oxide Donors | |
| | i. | Products or processes to increase NO synthesis in the liver |
| | | (increasing NO synthase activity) |
| | | Variety I |

| 1. | SIN-1 |
|---|---|
| 2. | Molsidamine |
| Variety II - nitrosylated forms of: | |
| 1. | N-acetylcysteine |
| 2. | Cysteine esters |
| 3. | L-2-oxothiazolidine-4-carboxolate (OTC) |
| 4. | Gamma glutamylcystein and its ethyl ester |
| 5. | Glutathione ethyl ester |
| 6. | Glutathione isopropyl ester |
| 7. | Lipoic acid |
| 8. | Cysteine |
| 9. | Cystine |
| 10. | Methionine |
| 11. | S-adenosylmethionine |

| | ii. | Products or processes to reduce the rate of NO degradation |
|---|---|---|
| | | in the liver |
| | iii. | Products or processes to provide exogenous NO or an |
| | | exogenous carrier or precursor which is taken up and |
| | | releases NO in the liver |
| f. | Antioxidants | |
| | i. | Vitamin E |
| | ii. | Vitamin C |
| | iii. | 3-morpholinosyndnonimine |
| g. | Glutathione increasing compounds | |
| | i. | N-acetylcysteine |
| | ii. | Cysteine esters |
| | iii. | L-2-oxothiazolidine-4-carboxolate (OTC) |
| | iv. | Gamma glutamylcystein and its ethyl ester |
| | v. | Glutathione ethyl ester |
| | vi. | Glutathione isopropyl ester |
| | vii. | Lipoic acid |
| | viii. | Cysteine |
| | ix. | Cystine |
| | x. | Methionine |
| | xi. | S-adenosylmethionine |

In light of the disclosure herein, one skilled in the art could readily determine if a particular candidate antagonist is a suitable antagonist by determining the method and dose of administration and performing toxicity studies according to standard methods (generally beginning with studies of toxicity in animals, and then in humans if no significant animal toxicity is observed). If the method and dose of administration do not result in acute toxicity, the antagonist is administered to the subject at the dose of administration and insulin resistance following treatment for at least three days in compare to pre-treatment insulin resistance. (Insulin resistance is assessed using the RIST test.) Where treatment results in increased insulin resistance without significant chronic toxicity (or having only modest chronic activity in a patient where untreated insulin resistance is life threatening), the antagonist is a suitable antagonist for that patient at the dose tested.

In some instances it will be desirable to manufacture and administer a pharmaceutical composition comprising a suitable acetylcholine esterase antagonist and another drug used in the treatment of diabetes.

In one embodiment acetylcholine esterase antagonists are preferably administered prior to each meal and having a duration of action about 4 to 6 hours.

For oral administration of acetylcholine esterase antagonists twice per day, each dose is preferably between 0.01 mg/kg body weight and 5 mg/kg body weight, when administered orally. In some embodiments an oral dose of between 0.05 mg/kg and 1.0 mg/kg will be desired. In some embodiments oral doses of between 0.15 and 0.7 mg/kg body weight will be desired. When the antagonist to be administered orally is pyridostigmine, in some embodiments dose of between 0.5 and 2.9 mg/kg body weight may be desired. Where the antagonist is specially targeted to the liver, the dose may be reduced accordingly.

For administration of acetylcholine esterase antagonists by twice-daily injection, a per-injection dose of between 0.001 and 0.05 mg/kg body weight may be desired. In some instances a per-injection dose of neostigmine of between 0.002 and 0.01 mg/kg body weight will be desired. In some instances a per-injection dose of an acetylcholine esterase antagonist of between 0.002 and 0.008 mg/kg body weight will be desired. Where the antagonist is targeted to the liver, dosages may be reduced accordingly.

The acetylcholine esterase antagonist may be administered so as to maintain a relatively constant level of the antagonist in the liver at all times. Alternatively, the antagonist may be administered to have antagonist concentrations peak when blood glucose is high, such as after a meal, so as to allow enhanced glucose uptake at that time. Where toxicity is a concern, it may be desirable to keep antagonist levels low until blood glucose levels become elevated above normal fasting levels. In many instances it will be desirable to administer the antagonist immediately before each meal. It will frequently be desirable to administer the antagonist so as to cause the acetylcholine concentration peak immediately prior to each meal and remain elevated for about 2-4 hours.

When administering or preparing to administer one or more acetylcholine esterase antagonists to a patient, reference should be had to toxicity studies performed according to standard techniques and relating to the compounds to be administered. In general, a patient should not receive a dose of one or more acetylcholine esterase antagonists sufficient to induce acute toxicity.

Patients should be monitored for signs of excessive exposure to acetylcholine esterase antagonists. These signs include (in typical order of appearance): salivation, sweating, decreased heart rate, bronchial constriction similar to asthma, and gastro intestinal upset including diarrhea and bladder incontinence.

In some instances it will be desirable to screen potential patients for HDIR prior to administering an acetylcholine esterase antagonist. One method of screening involves using the RIST methodology, described herein.

In one embodiment of the invention there is provided a kit containing an acetylcholine esterase antagonist in a pharmaceutically acceptable carrier together with instructions for the administration of the acetylcholine esterase antagonist to reduce insulin resistance in a patient. In one embodiment the kit further includes means to administer the acetylcholine esterase antagonist. Suitable administration means may be selected by one skilled in the art, depending on the route of administration desired.

In one embodiment of the invention there is provided a method of reducing insulin resistance in a mammalian patient comprising administering a suitable acetylcholine esterase antagonist.

In another embodiment of the invention there is provided a method of reducing insulin resistance in a mammalian patient suffering from inadequate levels of acetylcholine in the hepatic parasympathetic nerve synapses, the method comprising selecting a patient suffering from insulin resistance and administering a suitable acetylcholine esterase antagonist.

As used herein the phrase “suffering from inadequate levels of acetylcholine” means being in a condition where there is not sufficient acetylcholine to allow levels of signalling by the post-synaptic neuron sufficient to reduce insulin resistance to the level observed in an average healthy subject of the same gender, age, weight, fed-state, and blood sugar level as the patient.

In another embodiment of the invention there is provided a method of increasing glucose uptake by skeletal muscle of a patient suffering from suboptimal hepatic regulation of blood glucose levels, comprising selecting the patient and administering a suitable acetylcholine esterase antagonist.

Individuals suffering from insulin resistance who could in many cases benefit from treatment according to the methods described herein include those suffering from any one or more of: chronic liver disease, chronic hypertension, type II diabetes, fetal alcohol syndrome, gestational diabetes, and age-related insulin resistance and liver transplant recipients.

### EXAMPLES

### Example 1

Animal Studies

Male Sprague Dawley rats (250-300 g) were allowed free access to water and normal rodent food for 1 week prior to all studies. Rats were fasted for 8 hours overnight and fed for 2 hours before the start of study.

Surgical Preparation

Rats were anesthetized with pentobarbital-sodium (65 mg/ml, ip injection, 0.1 ml/100 g body weight). Animals were placed on a heated thermostatically controlled surgical table to maintain body temperature during surgery and the experimental procedure.

An extracorporeal arterial-venous shunt (the loop) was established between the right femoral artery and right femoral vein, according to a published, standard operating procedure developed in our laboratory (Xie et al., 1996). The loop allows for regular blood sampling of arterial blood throughout the experiment as well as infusion of intravenous drugs and monitoring of arterial blood pressure.

A tracheal breathing tube was inserted to ensure a patent airway and the jugular vein was cannulated for administration of supplemental anesthetic through out the study, and 10% w/vol glucose solution during the insulin sensitivity test procedure (rapid insulin sensitivity test, RIST). A laparotomy was performed and an indwelling portal venous catheter was inserted using a portal vein puncture technique. The portal catheter was used to administer the anticholinesterase agents directly to the liver.

Rapid Insulin Sensitivity Test (The RIST)

The Rapid Insulin Sensitivity Test (the RIST) is a euglycemic approach to test whole body glucose uptake in response to a low dose insulin challenge. It has been extensively validated against other standard approaches and has proven to be a sensitive, reliable and reproducible technique (Reid, et al., 2002).

Once surgery is completed, the rat is allowed to stabilize for approximately 30 minutes. At this point, blood samples (25 μl) are taken at regular intervals from the loop and analyzed for glucose concentration. Once a stable baseline glucose level is obtained, animals are given a 5 minute infusion of insulin (50 mU/kg) through the loop. Glucose levels are monitored every 2 minutes during and after the infusion of insulin. Exogenous glucose is infused into the jugular vein to prevent the hypoglycemic effect of insulin. Based on the glucose levels obtained from the regular blood sampling, the infusion rate of glucose can be adjusted to maintain the baseline euglycemia. Glucose infusion rates progressively increase as the effect of insulin reaches a maximum (at approximately 15 minutes into the test) and then progressively decrease as the effect of insulin wears off. Typically, the effect of insulin is complete by 35 minutes. The total amount of glucose infused during the RIST is considered the RIST index and is reported in terms of mg glucose infused/kg body weight of the subject.

Production of Insulin Resistance

As some degree of neural activation must remain for the anticholinesterase compounds to be effective, an atropine model of 75% blockade of HISS-dependent insulin resistance (HDIR) was developed. The dose of atropine used (5×10⁻⁶ mg/kg) was based on previously obtained dose-response data obtained in the rat. To this end, atropine was infused into the loop for 5 minutes. After allowing time to re-establish a stable blood glucose level, a RIST was performed to determine the degree of insulin resistance.

Reversal of Insulin Resistance with Neostigmine, an Anticholinesterase Agent

Neostigmine is an anticholinesterase agent that prevents the metabolism of acetylcholine, the neurotransmitter released from the parasympathetic nerves. After determining the degree of insulin resistance produced by atropine, neostigmine was constantly infused into the portal vein at a dose of 1 μg/kg/min. Neostigmine was infused for at least 30 minutes before a RIST was conducted to determine if this agent could reverse the insulin resistance.

Summary of Experimental Protocol

1. control RIST to determine insulin sensitivity
2. atropine infusion to produce a 75% block of HISS-dependent insulin resistance
3. post-atropine RIST
4. constant infusion of neostigmine into portal vein
5. RIST during neostigmine infusion
   Drugs

Human insulin (Humulin R) was obtained from Eli Lilly and Company. Atropine and neostigmine-bromide were obtained from Sigma Chemical Company. All drugs were diluted or dissolved in normal saline.

Results

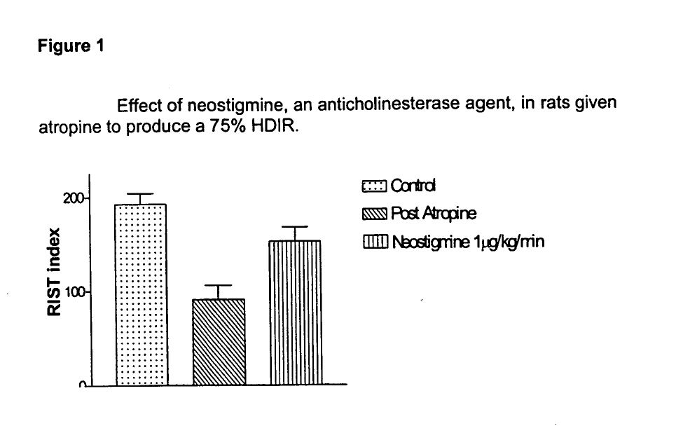

The average control RIST index was 192.4±11 mg/kg (n=3). Following the atropine-induced 75% HDIR, the RIST index was 90.5±15.2 mg/kg. The RIST index during the constant infusion of neostigmine (1 μg/kg/min, ipv) was increased to 152.6±15.2 mg/kg and is significantly increased from the blocked state. These data indicate that neostigmine is able to reverse the HDIR produced by atropine (FIG. 1).

### Example 2

Development of HDIR in a Model of Insulin Resistance Produced by High Sucrose Diets in Rats

It has been well documented that feeding rats a diet high in sucrose leads to a state of insulin resistance. The insulin resistance produced by this model has recently been shown to be HDIR.

Sucrose-fed Model of Insulin Resistance

Two approaches to sucrose-feeding were used in this investigation. In group one, 3 week old (weanlings), male, Sprague Dawley rats, were supplied for 12 weeks with a solid pellet diet in which 35% of all calories came from sucrose (solid diet group, Research Diets Inc.). In a second group, male, Sprague Dawley rats, approximately 6 weeks of age were provided free access to a 35% w/vol sucrose and water solution in addition to regular rodent pellet diet and normal drinking water for a 9 week period (liquid diet group).

Series 1: Assessment of HDIR in Sucrose Fed Rats

After the noted feeding period, both groups of rats were tested to determine the degree of HDIR that developed while on these diets. A control group consisted of rats fed only regular rodent diet.

Rats were fasted for 8 hours overnight and fed for 2 hours before the start of study. The surgical preparation was similar to that described above for normal rats treated with neostigmine except that no laparotomy was performed and no portal vein cannula was inserted. In brief, an arterial-venous shunt/loop was established, a tracheal breathing tube inserted and the jugular vein was cannulated.

Following a stabilization period and establishment of a baseline blood glucose level, a control RIST was conducted. Atropine was then administered (1 mg/kg) intravenously over 5 minutes to block the acetylcholine muscarinic receptors and produce a state of full HDIR. A second RIST was then conducted. The difference between the two RIST indexes indicates the degree of HDIR produced by sucrose feeding. For example, if the control RIST index and the post-atropine RIST index are similar, it suggests that the sucrose-feeding produces HDIR; if the difference is large, it suggest that sucrose-feeding is not producing HDIR.

Human insulin (Humulin R) was obtained from Eli Lilly and Company. Atropine was obtained from Sigma Chemical Company. Both drugs were diluted or dissolved in normal saline.

RIST indexes for the solid and liquid diet groups were 88±15 mg/kg (n=6) and 106±8 mg/kg (n=11), respectively and were not different. RIST indexes in the sucrose fed groups were significantly reduced from RIST indexes obtained from the control rats (n=9) fed only a regular rodent diet (197±10 mg/kg, **).

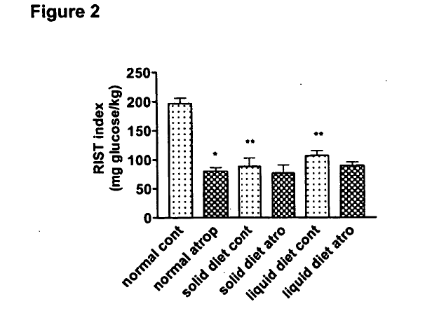

As shown in FIG. 2, following atropine administration to produce a full block of HISS release, RIST indexes were significantly reduced in the control rats (80±6 mg/kg, *), but were not significantly reduced in the sucrose fed groups (solid diet: 76±14 mg/kg; liquid diet: 89±7 mg/kg). These findings support the hypothesis that the insulin resistance observed following sucrose feeding is due to a reduction in HISS release/action, i.e., diminishment of the HISS-dependent component of insulin action.

### Example 3

Reversal of HISS-dependent Insulin Resistance in Sucrose-fed Rats using Anticholinesterase Agents

Since both forms of diet produced the same degree of HDIR, the model of sucrose feeding using the liquid diet was used to determine whether this HDIR was reversible with the anticholinesterase agent, neostigmine.

The model of insulin resistance produced by the 35% liquid sucrose diet (in addition to regular rodent food pellets and normal drinking water) was identical to the protocol described above for the assessment of HDIR in sucrose-fed rats.

Rats were fasted for 8 hours overnight and fed for 2 hours before the start of study. The surgical preparation was identical to that described above for sucrose-fed rats tested for HDIR. In addition, a laparotomy and portal vein cannulation were carried out. In brief, an arterial-venous shunt/loop was established, a tracheal breathing tube inserted and the jugular vein was cannulated. Following a laporotomy, the portal vein was cannulated.

After conducting a control RIST, neostigmine was infused into the portal vein for at least 30 before a second RIST was conducted to determine if this agent could reverse the insulin resistance. The doses of neostigmine were 1 and 2 μg/kg/min.

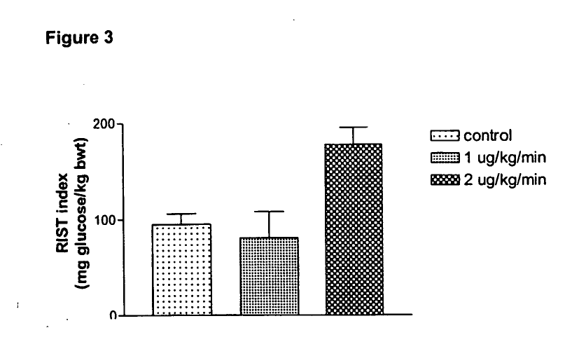

The control RIST index was 94.8±11.2 mg/kg and demonstrated that the liquid sucrose-fed rats were insulin resistant. As shown in FIG. 3, the dose of 1 μg/kg/min did not produce a reversal of insulin resistance (RIST index, 80.9±27.3 mg/kg) however, the dose of 2 μg/kg/min increased the RIST index to 178.0±17.7 mg/kg.

Thus, there has been provided a method of reducing insulin resistance.

References of Relevance to these Examples Include:

Xie, H. et al.: Am. J. Physiol. 270:E858 (1996); Sadri, P. et al.: Am. J. Physiol. 277:G1 (1999); Lautt, W. W. et al.: Can. J. Physiol. Pharmacol. 76:1 (1998); and Xie, H. et al.: J. Pharmacol. Toxicol. Meth. 35: 77-82 (1996).

1. Use of an acetylcholine esterase antagonist in the manufacture of a medicament useful in reducing insulin resistance in a mammalian patient suffering therefrom.

2. Use of an acetylcholine esterase antagonist in reducing insulin resistance in a mammalian patient suffering therefrom.

3. Use of claim 1 wherein the insulin resistance is at least partially the result of inadequate levels of acetylcholine in the patient's hepatic parasympathetic nerve synapses.

4. Use of an acetylcholine esterase antagonist in the manufacture of a medicament useful to increase skeletal muscle glucose uptake in a mammalian patient.

5. Use of an acetylcholine esterase antagonist to increase skeletal muscle glucose uptake in a mammalian patient.

6. Use of claim 1, wherein the patient suffers from suboptimal hepatic regulation of blood glucose levels.

7. Use of claim 1, wherein the acetylcholine esterase antagonist is at least one of donepezil, galanthamine, rivastigme, tacrine, physostigime, neostigmine, edrophonium, pyridostigmine, demecarium, pyridostigmine, phospholine, metrifonate, zanapezil, and ambenonium.

8. Use of claim 1 wherein the patient is a human.

9. A pharmaceutical composition comprising a suitable acetylcholine esterase antagonist and at least one other drug used in the treatment of diabetes.

10. The composition of claim 9 further including a pharmaceutically acceptable liver-targeting substance.

11. The composition of claim 9 wherein the antagonist is at least one of donepezil, galanthamine, rivastigme, tacrine, physostigime, neostigmine, edrophonium, pyridostigmine, demecarium, pyridostigmine, phospholine, metrifonate, zanapezil, and ambenonium.

12. The composition of claim 9, wherein the other drug is at least one of insulin, insulin analogues, sulfonylurea agents, tolbutamide, acetohexamide, tolazamide, chlorpropamide, glyburide, glipizide, glimepiride, biguanide agents, metformin, alpha-glucosidase inhibitors, acarbose, miglitol, thiazolidinedione agents (insulin sensitizers), rosiglitazone, pioglitazone, troglitazone, meglitinide agents, repaglinide, phosphodiesterase inhibitors, anagrelide, tadalafil, dipyridamole, dyphylline, vardenafil, cilostazol, milrinone, theophylline, sildenafil, caffeine, cholinergic agonists, acetylcholine, methacholine, bethanechol, carbachol, pilocarpine hydrochloride, nitric oxide donors, products or processes to increase NO synthesis in the liver, SIN-1, molsidamine, nitrosylated N-acetylcysteine, nitrosylated cysteine esters, nitrosylated L-2-oxothiazolidine-4-carboxolate (OTC), nitrosylated gamma glutamylcystein and its ethyl ester, nitrosylated glutathione ethyl ester, nitrosylated glutathione isopropyl ester, nitrosylated lipoic acid, nitrosylated cysteine, nitrosylated cystine, nitrosylated methionine, S-adenosylmethionine, products or processes to reduce the rate of NO degradation in the liver, products or processes to provide exogenous NO or an exogenous carrier or precursor which is taken up and releases NO in the liver, antioxidants, vitamin E, vitamin C, 3-morpholinosyndnonimine, glutathione increasing compounds, N-acetylcysteine, cysteine esters, L-2-oxothiazolidine-4-carboxolate (OTC), gamma glutamylcystein and its ethyl ester, glutathione ethyl ester, glutathione isopropyl ester, lipoic acid, cysteine, cystine, methionine, and S-adenosylmethionine.

13. The composition of claim 10, wherein the liver-targeting substance is at least one of albumin, bile salts and liposomes.

14. A kit comprising:
an acetylcholine esterase antagonist in a pharmaceutically acceptable carrier; and
instructions for the administration of the acetylcholine esterase antagonist to reduce insulin resistance in a mammalian patient.

15. The kit of claim 14 further comprising means to administer the acetylcholine esterase antagonist.

16. A method of reducing insulin resistance in a mammalian patient comprising administering a suitable acetylcholine esterase antagonist.

17. A method of amplifying the effect of the hepatic parasympathetic reflex on skeletal muscle insulin sensitivity comprising administering an acetylcholine esterase antagonist.

18. A method of increasing glucose uptake by skeletal muscle of a patient suffering from suboptimal hepatic regulation of blood glucose levels, comprising identifying the patient as suffering from suboptimal hepatic regulation of blood glucose levels and administering a suitable acetylcholine esterase antagonist.

19. A method of reducing insulin resistance in a mammalian patient suffering from inadequate levels of acetylcholine in the hepatic parasympathetic nerve synapses, said method comprising identifying the patient as suffering from inadequate levels of acetylcholine in the hepatic parasympathetic nerve synapses and administering a suitable acetylcholine esterase antagonist.

20. The method of claim 1 wherein the acetylcholine esterase antagonist is at least one of donepezil, galanthamine, rivastigme, tacrine, physostigime, neostigmine, edrophonium, pyridostigmine, demecarium, pyridostigmine, phospholine, metrifonate, zanapezil, and ambenonium.

21. The method of claim 1 wherein the acetylcholine esterase antagonist is targeted to the liver.

22. The method of claim 21 wherein the acetylcholine esterase is targeted to the liver using albumin.

23. The method of claim 21 wherein the acetylcholine esterase is targeted to the liver using a plurality of liposomes.

24. The method of claim 21 wherein the acetylcholine esterase is targeted to the liver using bile salts.

25. The method of claim 1 wherein the acetylcholine esterase is administered by intravenous administration.

26. The method of claim 1 wherein the acetylcholine esterase is administered by transdermal administration.

27. The method of claim 1 wherein the acetylcholine esterase is administered by oral administration.

28. The method of claim 1 wherein the acetylcholine esterase is administered by intra peritoneal administration.

29. The method of claim 1 wherein the acetylcholine esterase antagonist is administered by portal vein injection.

30. The method of claim 1 wherein the acetylcholine esterase antagonist is administered by immobilization of the acetylcholine esterase antagonist on a solid support and implantation of the support adjacent the patient's liver.

31. The method of claim 1 wherein the patient suffers from at least one of chronic liver disease, chronic hypertension, type II diabetes, fetal alcohol syndrome, gestational diabetes, age-related insulin resistance, and hepatic nerve damage.

32. The method of claim 1 wherein the patient is a human.

33. Use of claim 1, wherein the insulin resistance is hepatic insulin sensitizing substance-dependent insulin resistance.

34. The method of claim 1 wherein the insulin resistance is hepatic insulin sensitizing substance-dependent insulin resistance.

35. Use of claim 2 wherein the insulin resistance is at least partially the result of inadequate levels of acetylcholine in the patient's hepatic parasympathetic nerve synapses.

36. Use of claim 2 wherein the patient suffers from suboptimal hepatic regulation of blood glucose levels.

37. Use of claim 4 wherein the patient suffers from suboptimal hepatic regulation of blood glucose levels.

38. Use of claim 5 wherein the patient suffers from suboptimal hepatic regulation of blood glucose levels.

39. Use of claim 2 wherein the acetylcholine esterase antagonist is at least one of donepezil, galanthamine, rivastigme, tacrine, physostigime, neostigmine, edrophonium, pyridostigmine, demecarium, pyridostigmine, phospholine, metrifonate, zanapezil, and ambenonium.

40. Use of claim 4 wherein the acetylcholine esterase antagonist is at least one of donepezil, galanthamine, rivastigmne, tacrine, physostigime, neostigmine, edrophonium, pyridostigmine, demecarium, pyridostigmine, phospholine, metrifonate, zanapezil, and ambenonium.

41. Use of claim 5 wherein the acetylcholine esterase antagonist is at least one of donepezil, galanthamine, rivastigme, tacrine, physostigime, neostigmine, edrophonium, pyridostigmine, demecarium, pyridostigmine, phospholine, metrifonate, zanapezil, and ambenonium.

42. Use of claim 1, wherein the insulin resistance is hepatic insulin sensitizing substance-dependent insulin resistance.
